(19) United States
(12) Patent Application Publication
(10) Pub. No.: US 2013/0028959 A1
(43) Pub. Date: Jan. 31, 2013

(54) Liposomes for Preventing the Spread of HIV

(71) Applicants: Nikita Kiran Malavia, San Diego, CA (US); Daniel S. Kohane, Newton, MA (US)

(72) Inventors: Nikita Kiran Malavia, San Diego, CA (US); Daniel S. Kohane, Newton, MA (US)

(73) Assignees: Massachusetts Institute of Technology, Cambridge, MA (US); Children's Medical Center Corporation, Boston, MA (US)

(21) Appl. No.: 13/516,043

(22) PCT Filed: Dec. 16, 2010
(86) PCT No.: PCT/US2010/060790

§ 371 Date: Jun. 14, 2012

Related U.S. Application Data
(60) Provisional application No. 61/287,158, filed on Dec. 16, 2009.

(51) Int. Cl. A61K 9/127; A61K 9/133; A61K 38/46; A61P 31/12; A61P 31/18; A61K 38/17; A61K 39/395; B82Y 5/00

(52) U.S. Cl. 424/450; 514/3.7; 424/130.1; 424/94.6; 977/773; 977/801; 977/907; 977/915; 977/797

(57) ABSTRACT

Formulations for preventing the sexual transmission of the HIV virus are provided. In one embodiment, the formulations contain un-conjugated liposomes whose physicochemical properties allow binding to the HIV virus. The liposomes are made up of natural or synthetic lipids, alone or in combination. Preferably, the liposomes are made entirely of cardiolipin. In other embodiments the liposomes are modified to contain Hgands which bind HIV. The method for preventing the sexual transmission of the HIV virus includes self-administration of a formulation containing an effective amount of liposomes which bind to the HIV virus to the surface of a mucosal membrane prior to intercourse.

### FIELD OF THE INVENTION

This invention relates to formulations comprising liposomes, particularly to topical formulations, for preventing the transmission viral infections, more particularly sexual transmission of the human immunodeficiency virus (HIV).

### BACKGROUND OF THE INVENTION

Recent statistics indicate there are 33.2 million people infected with HIV-1 with over 16,000 new infections occurring every day (Fauci, Clin Infect Dis, 45(Suppl 4):S206-12 (2007)). Current treatments are susceptible to the development of drug resistance, and have associated toxicities. Furthermore, poverty and lack of access to medical care place some of the crucial advances in the treatment of Acquired Immune Deficiency Syndrome (AIDS)—such as antiretroviral drugs and combination therapies beyond the reach of many in the developing world. Consequently, a portion of the scientific community has turned to prevention. There is no safe and clinically effective vaccine to protect against this disease (Letvin, Nat Rev Immunol, 6(12):930-9 (2006)). An example of a recent promising approach is the combinatorial vaccine that was able to cut the risk of infection by more than 31% in almost 16,000 participants (Berkhout, et al. Retrovirology, 6(1): p. 88 (2009)).

An alternative approach is seen in the recent trend toward the development of topical vaginal microbicides to inhibit transmission of sexually transmitted diseases including Human Immunodefiency Virus 1 (HIV-1). This approach evolves from the fact that women in the developing world are greatly affected by HIV, with sexual transmission being the primary mode of infection. Such microbicides have to be inexpensive, effective, safe, stable and widely acceptable to be used (Moore, N Engl J Med, 352(3):298-300 (2005)). So far, most of the microbicide candidates have failed, some miserably (Klasse, et al., PLoS Med, 3(9):e351 (2006)). The use of vaginal microbicides is also appealing in that it empowers women to easily instigate prophylaxis in social contexts where more conventional forms of prophylaxis might be beyond their control. Thus, there remains an immediate and urgent need for cheap, safe and effective means of preventing the spread of HIV.

It is an object of the present invention to provide formulations for the prevention of HIV transmission that are inexpensive and easy to apply.

It is another object of the present invention to provide a method for preventing the sexual transmission of the HIV virus.

### SUMMARY OF THE INVENTION

Formulations for preventing the sexual transmission of the HIV virus are provided. The formulations contain an effective amount of liposomes which act as decoys causing the HIV virus to attach to the liposomes instead of host cells. In one embodiment, the formulations contain unconjugated liposomes whose physicochemical properties allow binding to the HIV virus. In some embodiments the liposomes are modified to have a ligand that binds to HIV. For example, soluble CD4 or CD4 mimic peptides can be conjugated to the surface of liposomes. In this embodiment the CD4 or CD4-like peptides binding the HIV virus and preventing it from infecting cells. Antibodies that recognize and bind to peptides on the HIV glycoprotein coat can also be conjugated on the liposomal surface. The liposomes are made up of natural or synthetic lipids, alone or in combination. The liposomes may be made entirely of one type of lipid or of a combination of lipids. In a preferred embodiment, the liposomes are made entirely of cardiolipin.

Also provided is a method for preventing the sexual transmission of the HIV virus which includes self-administration of a formulation containing an effective amount of liposomes which bind to the HIV virus. The formulation is applied to the surface of a mucosal membrane prior to intercourse. The formulations can also be applied prior to penetration of non-sexual organs. In a preferred embodiment, the formulation is applied to the vagina prior to sexual intercourse.

### BRIEF DESCRIPTION OF THE DRAWINGS

FIG. 1 is a schematic showing a proposed mode of action of the disclosed formulation.

FIG. 2 is a schematic showing recombinant soluble CD4 or CD4 mimic peptides on the surface of a liposome which are bound to a viral particle.

FIG. 3 is a schematic showing liposome production.

FIG. 4 is a schematic showing the single round infection assay procedure.

FIG. 5A is a graph showing the viral infection using the YU2 virus isolate. FIG. 5B is a graph showing the viral infection using the AMLV virus isolate (♦=DSPG; ▪=PC; ▴=PS; x=Cl; *=DMPG; =PI; =PBS).

FIG. 6 is a bar graph showing the cytotoxicity of liposomal formulations at various concentrations after two days on HeLa cells (PC, PI, CL, DMPG, PS, DOTAP).

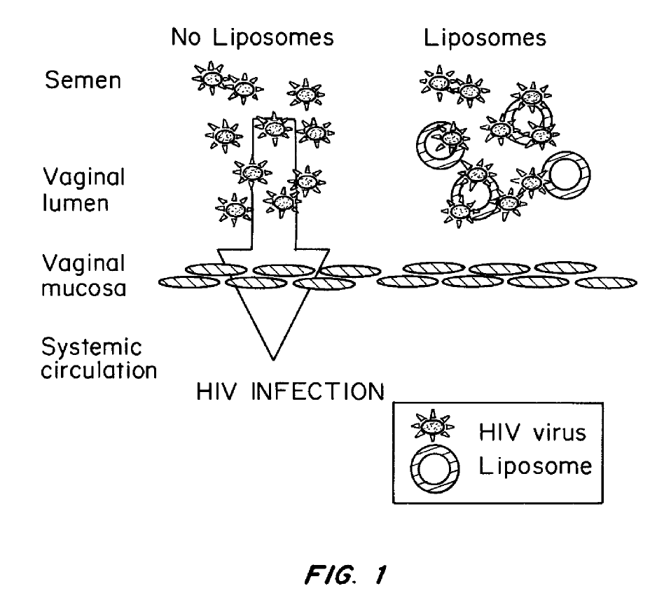

### DETAILED DESCRIPTION OF THE INVENTION

### I. Liposome Formulations

(a) Liposomes

Liposomes are spherical, self-enclosed vesicles formed by one or more bilayers of natural or synthetic lipids or combinations thereof, with aqueous phases inside or in between layers of lipids (Torchilin, Nat Rev Drug Discov, 4(2):145-60 (2005). They are made up of one or many concentrically arranged lipid bilayers constituting an envelope. The lipid vesicles comprise either one or several aqueous compartments delineated by either one (unilamellar) or several (multilamellar) phospholipid bilayers (Sapra, et al., Curr Drug Deliv 2:369-81 (2005)).

The basic components of liposomes are amphiphilic compounds having clearly separated hydrophilic and hydrophobic centers. Hydrophilic components of lipids in the bilayer are directed towards aqueous phases (external and internal), whereas the hydrophobic components of both lipid layers are directed towards one another, forming the internal layer of a membrane. The principal components of liposomes phospholipids, triglycerides, and cholesterol—are also natural components of biological membranes. The compounds most often used for the preparation of liposomes preparation are phospholipids; however, liposomes have also been prepared from single-chain natural or synthetic amphiphiles, such as lipid acids, saponins and/or detergents. Liposomes can also be prepared by derivatizing phospholipids with a biocompatible, hydrophilic polymer as described in U.S. Pat. No. 6,930,087 to Baru et al. Liposomes can be used to encapsulate hydrophilic, hydrophobic and amphiphilic materials. Hydrophilic materials are encapsulated in the internal aqueous phase while hydrophobic materials are incorporated into the lipid bilayer and amphiphilic materials are absorbed onto the double lipid membrane. Moreover, charged active substances may be attached to the surface of the bilayer.

The liposome includes a ligand for a virus, preferably for HIV. The ligand can be a lipid, modified lipid, protein, or polypeptide. In one embodiment, the ligand is CD4 or an HIV binding fragment thereof. In the preferred embodiment, the liposome formulations disclosed herein reduce or prevent the spread of viral infection as a function of the physicochemical properties of the liposomes. Physicochemical properties that can be manipulated to enhance binding of the liposome to the virus include charge, hydrophobicity, lipid composition, liposome size and protein content.

In a preferred embodiment, the liposomes are negatively charged. The anionic charge enhances binding to the positively charged amino acids on the virus. The liposomes are preferably small in size, with small size increasing binding by increasing the surface area-to-volume ratio of the particles. The size of the liposome can be than 100 μm in diameter, more preferably, between 50 nm and 4 μm in diameter.

Melting temperature of the lipids (Tₘ) directly affects liposomal particle leakiness, also affecting the liposomal ability to fuse with virus. The higher the melting temperature—particularly with respect to body temperature (37° C.), the less fluid/permeable and more stable the formulations will be. The permeability of liposomes varies with the increase or decrease of the distance between the lipid molecules of the bilayer (Dhoot, et al., J Pharm Sci, 92(3):679-89 (2003)). The lipids can be selected such that the liposomes are composed of lipids with high melting temperatures.

In some embodiments, the liposomes are modified to attach CD4 and CD4-derived peptides or other ligands of HIV.

Liposome size can be varied by repeated freeze thaw cycles of incubating liposomes in liquid nitrogen and at temperature above the Tₘ.

(i) Lipids

Natural lipids and synthetic derivatives of lipids can be used to make the liposomes. Examples include derivatives of phosphatidylglycerol (e.g. Distearoylphosphatidylglycerol (DSPG) and Dimyristoylphosphatidylglycerol (DMPG)), Cardiolipin (CL), Phosphatidylinositol (PI), Phosphatidylglycerol (PG), Phosphatidylserine (PS), Phosphatidylethanolamine (PE), Phosphatidic acid (PA) and Phosphatidycholine (PC). Other applicable synthetic lipids include oleoyl, myristyol and stearoyl synthetic derivatives of PI, PS, PE, PA and PC. Additional molecules include the lipid-like molecules disclosed in Akinc, et al., Nat. Biotechnol., 26(5):561-9 (2008). In a preferred embodiment the liposome is made of cardiolipin from about 10, 20, 50 or 75 percent cardiolipin. In a more preferred embodiment, the liposome is made entirely cardiolipin.

(ii) Additional Liposomal Agents

Cholesterol, which is present in all cell membranes, can be included in the liposomes to enhance liposome fusion with the virus, based on its effect on HIV virus fusion with cells (Viard, et al., J Virol, 76(22):11584-95 (2002)). The cholesterol/phospholipid molar ratio is higher in the HIV envelope than in host cell membranes (Raulin, Prog Lipid Res, 41(1):27-65 (2002)). The proportion of cholesterol can be added from 0-20% of the total lipid content.

Polyethylene glycol (PEG) coating has been reported to prevent non-specific attachment to cells e.g. macrophages (Miller, et al., Biochemistry, 37(37): p. 12875-83. (1998)). In these embodiments PEG with average molecular weights ranging from 2000-5000 and at concentrations from 0-10% of the total lipid content can be used.

(b) Other Active Agents

The formulation may additionally contain a microbicide, a spermicide and/or any other drug. These may include free microbicide(s), spermicide(s) and/or any other drug which chemically destroys pathogens coming into contact with the mucosa and also reduces or eliminates toxicity of the microbicides, spermicides and/or other drug to epithelial cells. Microbicides, spermicides and/or any other drug may be released from the gel or they may get in situ on pathogens or infected cells.

The formulations may additionally contain inhibitors of HIV protease and reverse transcriptase (alone or in combination). These inhibitors are preferably encapsulated within the liposomes.

(c) Carriers and Excipients

The liposomes may be administered in a suitable carrier such as an ointment, gel, paste, lotion, sponge, or spray, soft gelatin capsules. The liposomes may be administered in a paste or gel which is placed in a soft gelatin capsule. In a preferred embodiment, the liposomes are administered in a gel.

Standard excipients that can be included in the disclosed formulations are gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars and starches.

In one embodiment, the gel is made of a polaxamer polymer. Poloxamer 407 is a block copolymer of polyoxyethylene and polyoxypropylene in a 7:3 weight ratio with an average molecular weight of 12500. One important characteristic of this block copolymer is its ability to form a thermoreversible gel. The transition from the liquid state at low temperature to the gel state at body temperature (the phase transition temperature being dependent, in part, on the concentration of the gel and on the ionic strength) allows a number of medical applications including topical applications. The gel formulations of poloxamer 407 of any suitable concentration (w/w) can be prepared, and more particularly those between about 10 and 50% w/w are used.

(d) Dosage Forms

The liposomes described herein can be formulated into any dosage form suitable for topical administration on a mucosal surface. Suitable dosage forms for topical administration include ointments, pastes, creams, lotions, gels, ointments, pastes, solutions, sprays or foams. Methods for making these dosage forms are well known in the art. See, for example, Ansel, Popovich, and Allen, Pharmaceutical Dosage Forms and Drug Delivery Systems 6^{th} Ed., Williams and Wilkins, 1995.

1. Creams, Pastes, and Ointments

i. Creams

Creams are generally characterized as semisolid dosage forms formed from an oil-in-water emulsion, a water-in-oil emulsion, or an aqueous microcrystalline dispersion. Creams are generally less viscous and lighter than ointments. Creams are considered to have greater esthetic appeal than ointments or pastes due to their non-greasy character.

ii. Ointments

Ointments are prepared by mixing one or more active agents in an ointment base. The ointment base is semisolid and can be either hydrophobic or hydrophilic. Suitable ointment bases include hydrocarbon bases, such as petrolatum, white petrolatum, yellow ointment, and mineral oil; absorption bases, such as hydrophilic petrolatum, anhydrous lanolin, lanolin, and cold cream; water-removable bases, such as hydrophilic ointments; and water-soluble bases, such as polyethylene glycol ointments. Suitable bases include, but are not limited to, petrolatum, mineral oil, vegetable oils, and combinations thereof,

iii. Pastes

Pastes contain more solid materials than ointments and are therefore stiffer and less penetrating. Because of their large percentage of solids, pastes are generally more absorptive and less greasy than ointments prepared from the same components.

2. Lotions

While creams, pastes, and ointments are classified as semisolid preparations, lotions are characterized as liquids. Lotions are generally suspensions of solid materials in an aqueous vehicle, although certain emulsions and even some true solutions have been designated as lotions due to their appearance or application. Lotions may be preferred over semisolid formulations due to their non-greasy character and their increased spreadability over large areas. Lotions typically contain finely powdered substances that are insoluble in the dispersion medium and are suspended through the use of suspending agents and dispersing agents. Other lotions can have as the dispersed phase liquid substances that are immiscible with the vehicle and are usually dispersed by means of emulsifying agents or other suitable stabilizers,

3. Foams

Pharmaceutical foams are pressurized dosage forms containing one or more active ingredients that, upon valve actuation, emit a fine dispersion of liquid and/or solid materials in a gaseous medium. Foam formulations are generally easier to apply, are less dense, and spread more easily than other topical dosage forms. Foams may be formulated in various ways to provide emollience depending on the formulation constituents.

Foams may contain an emulsion. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

The oil phase can be prepared by mixing together the surfactant(s) and emulsifier(s) and melting. The aqueous phase is prepared separately by dissolving the preservatives in water with heating. The aqueous phase is added to the oil phase with continuous high shear mixing to produce a milky emulsion. The emulsion is cooled and the pH is adjusted by the addition of a buffer. The active agent can be either pre-dissolved in aqueous or organic phase or suspended/dispersed in the final emulsion.

Foams generally contain a pharmaceutically acceptable propellant. Suitable propellants include, but are not limited to, hydrofluoroalkanes, hydrofluorocarbons, volatile alcohols, hydrocarbon gases, and combinations thereof. Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable.

The emulsion concentrate is placed in any suitable vaginal foam applicator.

### II. Methods of Making Liposome Formulations

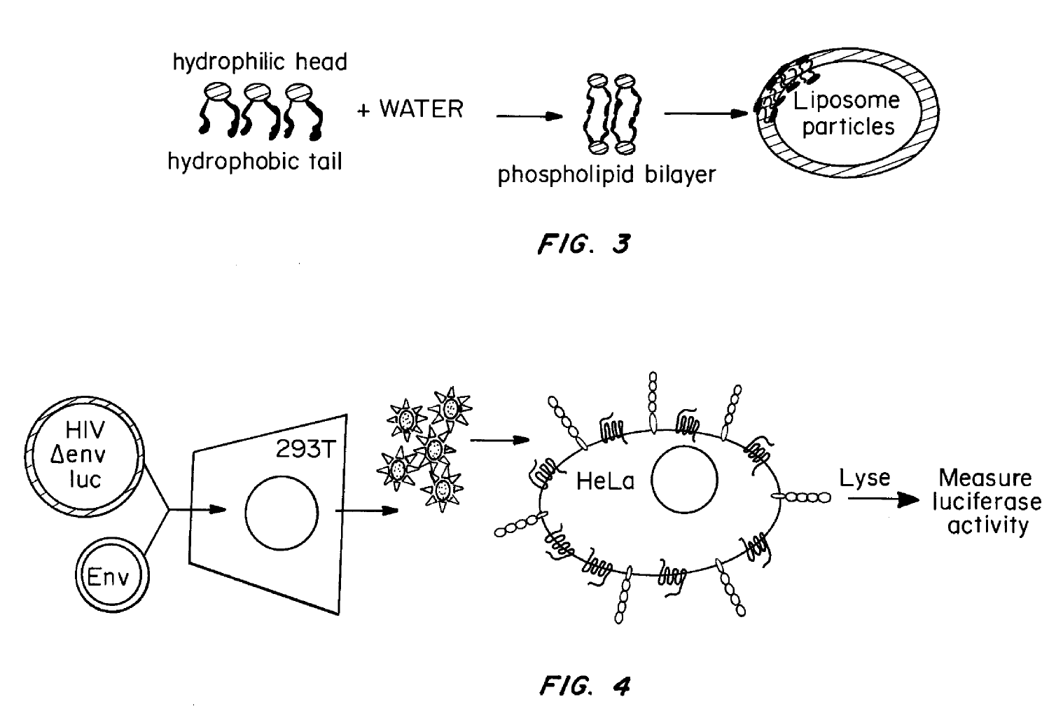

A schematic for making the liposomes using various lipids as disclosed herein is shown in FIG. 3. Liposomes can be prepared using methods known in the art or as discussed in the Examples. Other known organic solvents can be used to make the formulations, or reverse phase evaporation can be used.

A library of liposomes can be prepared with varying physicochemical properties and tested for their ability to bind the HIV virus. Freeze-fracture analysis is performed to characterize the morphology of the liposomes. Stability assays are performed as described (Epstein-Barash, et al., Proc Natl Acad Sci USA, 106(17):7125-30 (2009)).

CD4 and CD4 Derived Peptides Conjugation

Using the service of GenScript Corp., CD4-derived peptides are synthesized as described in Perdomo, et al., Proc Natl Acad Sci USA, 105(34):12515-20 (2008). The CD4 protein molecule or peptides are conjugated using known methods such as sulihydryl-maleimide chemistry, N-hydroxysuccinimide (NHS), 1-ethyl-3-(3-dimethylaminopropyl)-carbodiimide (EDC) chemistry.

Liposomes can be formulated to contain fluorescent lipids. Fusion between fluorescent liposomes and cells can then be easily monitored by confocal microscopy and/or transfer of fluorescent dye to cells (Kheirolomoom, et al., J Control Release, 2007. 118(3):275-84 (2007); Zhukovsky, et al., Biophys J, 91(9):3349-58 (2006)).

Fluorescence microscopy can be utilized to confirm CD4 attachment on the surface of liposomes. Briefly, one component of the liposomes is labeled with rhodamine lipids (Avanti Polar lipids). FITC (green fluorescence) conjugated CD4 antibody (R&D systems) is added to the CD4 conjugated liposomes as per manufacturer's protocol and subsequently imaged using a fluorescence microscope, with appropriate antibody only and no antibody controls. Quantification of the fluorescence from the conjugated liposome versus untargeted and secondary alone control is used to confirm conjugation of CD4 on liposomes.

Liposomes conjugated to scrambled sequences or putatively irrelevant proteins are other controls that can be used. Additional controls include competitive binding assays between labeled gp120 and conjugated vs. unconjugated liposomes (Madani, et al., Structure, 16(11):1689-701 (2008); Schon, et al., Biochemistry, 45(36):10973-80 (2006)). Other methods are known in the art (Xiang, et al., J Virol, 79(10):6068-77 (2005); Madani, et al., J Virol, 2004. 78(7):3742-52 (2004) and Haim, et al., 5(4):e1000360 (2009)).

The Ability of these Engineered Liposomes to Inhibit Infection Successful candidates are identified based on the following criteria: (i) less than 1% infection using the single round infection assay with various HIV-1 isolates (e.g. YU2) at particle concentrations of 100/μl or less (ii) non toxic to cells at concentrations when infection is <1% determined by toxicity assay.

Murine models can be used to assess formulations for toxicity, biocompatibility, and the ability to inhibit infection in vivo. An example of a transgenic mouse model susceptible to HIV infection is described in Browning, et al., PNAS, 103(38):13938-43 (2006). Healthy intact vaginal epithelium provides a significant barrier against infection. Thus any microbicide candidate that may comprise the vaginal barrier could cause a major increase in susceptibility. Biocompatibility testing of disclosed formulations can be performed in the murine vagina. For assays of infection prevention, Depo-provera can be used to thin the animal's vaginal epithelium prior to instilling HIV inocula and formulations.

Female Swiss mice are pretreated with Depo-Provera, which eliminates the layer of dead cells that otherwise protects the vagina (Vargas, et al., J Infect Dis, 199(10):1546-52 (2009)). One group is not treated with Depo-provera for biocompatibility analysis. Animals receive a test solution (conjugated or un-conjugated liposomal formulations in phosphate buffered saline [PBS] solution, HIV virus) transvaginally using a Wiretol pipette under isofurane/oxygen anesthesia. Mice which do not receive Depo-provera and mice treated with PBS are only used as controls. For biocompatibility and in vivo distribution testing, the mice are euthanized at appropriate time points (8 h and 24 h, 2 days) and the reproductive tract excised and immersed in formalin for fixation. Serial 10 μm sections are made and stained for histological analysis. In addition, another group receives fluorescently labeled liposomes for whole-animal fluorescent imaging studies using the IVIS® system. This small animal imaging system allows for monitoring of particle distribution in real time (the time frame in these mice will be 1 h, 2 h, 4 h, as above). These mice are sacrificed at 24 hours and the number of fluorescent particles in the vagina compared to other organs.

Histological Analysis

Sectioned tissues are stained with hematoxylin-eosin (H&E) and for epithelium integrity markers (e.g. ZO-1, cytokeratin) and examined by microscopy. The distance from the surface to the basement membrane is used to determine epithelium thickness using a Nikon E800 epifluorescence microscope.

Toxicity

At the same time intervals after instillation of liposomes or PBS as discussed above, 20 μl of YOYO-1 [(Invitrogen) a cell-impermeant dye that becomes highly fluorescent in contact with dsDNA in the nuclei of cells whose membranes are disrupted] is instilled in the vagina. The vagina is lavaged with PBS to remove excess dye. At the end the animal is sacrificed and gross images of the entire vagina taken using a Nikon E800 epifluorescence microscope.

Cytokine Assay

To detect increases in mouse-specific cytokines in the vaginal vault after exposure to the formulations, animals which have been treated with optimized liposomes are lavaged with PBS. These samples (and controls) are analyzed by the Bio-plex system (Luminex Corp.) which permits assaying of up to 100 ELISA (Enzyme-linked immunosorbent assay) assays from one sample. The assay detects any unwanted increase in cytokine levels beyond baseline in the ‘treated with liposomes’ condition.

Infection Model

Animals are treated with a Liposomal formulation or saline. Twenty minutes later, a low (1 Infectious dose₅₀ (ID₅₀)) and high 10 ID₅₀ dose of viral inoculum is introduced using a Wiretol tip (Catalone, et al., Antimicrob Agents Chemother, 48(5): 1837-47 (2004)). Lavages with 50 μl of medium are obtained 3 days after the inoculum as this is when the animal reached maximum viral shedding. The lavages are spun down and the supernatants are collected and exposed to human foreskin fibroblast cells. 48 hours later, the fibroblast cells are examined for cytopathic effects by light microscopy. Survival is assayed by the MTT assay.

Protection Assay

Non-toxic formulations are then tested in the infection model. Here the high and low viral inoculum is mixed with liposomal formulations and delivered to the mouse vagina. Infection and susceptibility assays are performed as described (Cone, et al., BMC Infect Dis, 2006. 6:90 (2006)).

### III. Methods of Using the Liposome Formulations

The liposome formulations described herein can be applied directly to any mucosal membrane prior to sexual intercourse. The mucosal surface may be any surface that comes in contact with bodily fluid containing the virus, such as the mouth, arms, anus, or vagina. In a preferred embodiment, the formulation is applied on the vaginal or cervical mucosae. The compositions may be in the form of a solution, topical ointment, cream, foam, suppository, ovule, or an aerosol.

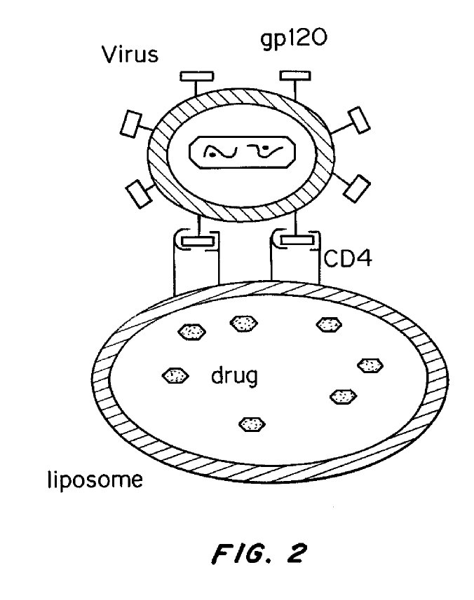

The HIV virus is about 120 nm in size and is positively charged. The gp120 knob-like structure on the virus binds to the CD4 molecule on human cells to initiate fusion and infection. The physicochemical properties of the liposome formulations disclosed herein are manipulated to enable binding of the HIV-virus to the liposomes. In one embodiment, the liposome contains a ligand for the HIV virus. The ligand may be one or more lipids or modified lipids in the liposome, or a protein or a peptide. In one embodiment, the liposomes are engineered to resemble cell surfaces, by attaching CD4 and CD4-like peptides onto the surface of the liposomes. These peptides bind to the gp120 knob-like structures of the virus and prevent the virus from infecting cells (FIG. 2).

The present invention will be further understood by reference to the following non-limiting examples.

### EXAMPLES

Liposome formulations were made from lipids shown in Tables 1-3.

TABLE 1
Surface charge and hydrophobicity of some lipids used to
make liposomes

| | | Relative | Degree of | Actual |
|---|---|---|---|---|
| Group # | Name of lipid | charge | hydrophobicity^{a} | Charge |
| 1 | Cardiolipin (CL) | −−− | ++++ | − |
| 2 | Dioleoylphos- | −− | ++ | − |
| | phatidylglycerol | | | |
| | (DOPG) | | | |
| 3 | Phosphatidylinositol | −− | + | − |
| | (PI) | | | |
| 4 | Phosphatidylserine | − | + | − |
| | (PS) | | | |
| 5 | Phosphatidylcholine | No | ++ | −/+ |
| | (PC) | charge | | (zwitterions) |
| 6 | Dioleoyltrimethyl- | + | ++ | + |
| | ammonium | | | |
| | propane | | | |
| | (DOTAP) | | | |

^{a}Derived from the number of hydrophobic tails.

Lipids were dissolved in chloroform solution, dried under a steady stream of nitrogen gas, resuspended in a suitable organic solvent (selected based on the lipids used) by vortexing and quick frozen in liquid nitrogen. The solvent was evaporated till completely dry and then lyophilized overnight, following which the lipids were resuspended in phosphate buffered saline (PBS) above their melting temperature (Tₘ) at a final lipid concentration of 10 mg/ml.

The melting temperature of lipids and lipid leakiness was determined for three different lipids as shown in Table 2.

TABLE 2
Melting temperature of lipids/leakiness of lipids

| | | Degree of |
|---|---|---|
| Name of lipid | Tm (C.) | leakiness |
| Dioleoylphosphatidylglycerol | −18 | *** |
| (DOPG) | | |
| Dimyristoylphosphatidylglycerol | 23 | ** |
| (DMPG) | | |
| Distearoylphosphatidylglycerol | 55 | * |
| (DSPG) | | |

Liposome size was measured using a Multisizer™ 3 Coulter Counter® (Beckmann Coulter) and surface charge (zeta potential in mV) by Quasi-elastic laser light scattering with a ZetaPALS dynamic light scattering detector (Brookhaven Instruments); the latter ca also be used to size particles <1 μm in diameter. The sizes of liposomes that can be made from different lipids are shown in Table 3.

TABLE 3
Average size and zeta potential of liposomes

| Lipid | Average Size | Zeta Potential |
|---|---|---|
| Only | (microns) | (mV ± SD |
| CL | 4 | −88.8 ± 2.7 |
| PI | 2 | −84.6 ± 5.2 |
| PS | 4 | −59.1 ± 3.3 |
| PC | 5 | −5.8 ± 0.6 |
| DOTAP | 3 | 74.9 ± 3.1 |
| DMPG | 4 | −59.2 ± 1.0 |
| DSPG | 3 | −14.5 ± 4.5 |

HIV Inhibition Assay

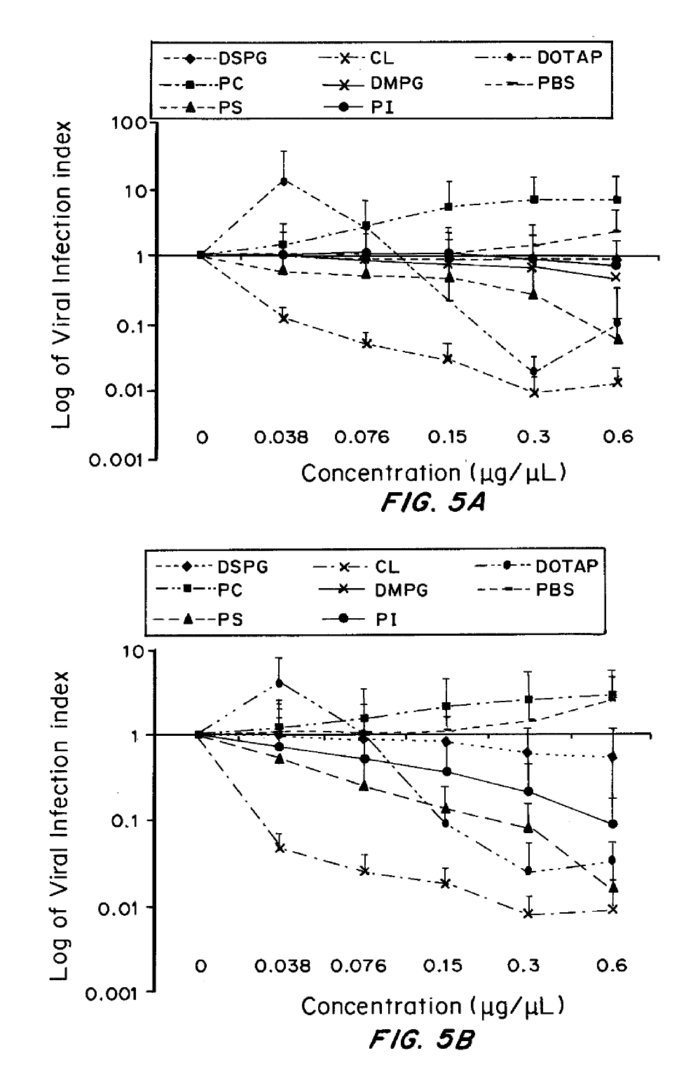

Different liposomal formulations were screened for their ability to inhibit HIV-1 infection using the single round infection assay as depicted in FIG. 4. 293T cells were genetically modified to manufacture luciferase viruses that were incubated with liposomes. Target cells expressing CD4 and CCR5 were seeded in 96-well luminometer-compatible tissue culture plates 24 h before infection. On the day of infection liposomes at various concentrations were incubated with luciferase expressing recombinant viruses at 37° C. for 30 min. The mixtures were added to the target cells and incubated for 2 h at 37° C. Following 48 h incubation, the media was removed and the cells lysed by the addition of passive lysis buffer (Promega) (Schon, et al., Biochemistry, 2006. 45(36): p. 10973-80 (2006); Madani, et al., Structure, 16(11): p. 1689-701 (2008). An EG&G Berthold Microplate Luminometer LB 96V was used to measure the luciferase activity of each well after the addition of luciferin buffer (BD Pharmingen). The assay was carried out using two s HIV-1 isolates, YU2 (FIG. 5A), and a non-specific murine leukemia virus (AMLV) (FIG. 5B). The viral infection index was calculated as follows.

Viral Infection Index=luciferase activity of virus with liposomes/Luciferase activity of virus alone

Cytotoxicity Assay

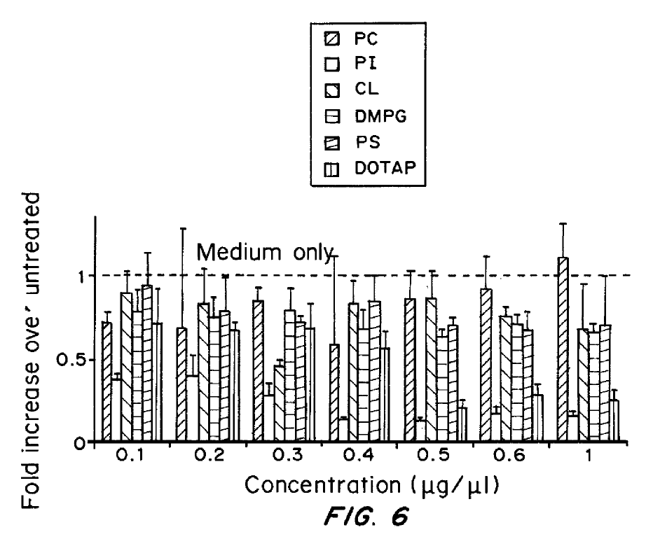

Cytoxicity of liposomal formulations was tested in uninfected HeLa cells using a Live Dead assay and a commercially available MTT assay performed following the manufacturer's instructions. Briefly, liposomes were exposed to HeLa cells at various lipid concentrations for 2 days. After this incubation period, the MTT cytotoxicity test was performed. The absorbance in the treated condition was normalized to the medium only condition (untreated) (FIG. 6).

Infection of HeLa cells by HIV-1 (YU2) and another model virus (AMLV) occurs in more than 100% of cases when phosphate buffered saline is added (as a control treatment) to the culture medium. Negatively charged cardiolipin liposomes greatly reduces the rate of infection by HIV virus YU2 and AMLV by more than 100 fold (FIG. 5A and FIG. 5B). The decrease in infectivity is not due to cytotoxicity to HeLa cells (FIG. 6).

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

1. A composition for preventing the spread of a viral infection, wherein the composition comprises liposomes comprising a ligand for the virus.

2. The composition of claim 1 wherein the liposomes are multilamellar, unilamellar or multivesicular.

3. The composition of claim 2, wherein the liposomes are made from lipids selected from the group consisting of cationic lipids, anionic lipids, lipids that are neutral in charge, lipids that are zwitterionic, and combinations thereof.

4. The composition of claim 1, wherein the liposomes have a size ranging from 1 to 100 micrometers.

5. The composition of claim 4, wherein the liposomes have a size range selected from the group consisting of 10 to 100 nm, 100 nm to 999 nm and 10 to 100 micrometers.

6. The composition of claim 3, wherein the liposomes comprise cardiolipin.

7. The composition of claim 6 where the liposomes comprise 10%, 25%, 50%, 75% or 100% cardiolipin.

8. The composition of claim 3, wherein the ligand is a surface ligand that enhances binding to the virus.

9. The composition of claim 1, wherein the liposomes further comprise one or more bioactive agents selected from the group consisting of drugs and microbicides.

10. The composition of claim 9 wherein the drug or microbicide is selected from the group consisting of nucleases, antibodies, spermicides and spermicidals.

11. The composition of claim 1, wherein the liposomes are in a dosage form selected from the group consisting of ointments, gels, pastes, lotions, sponges, sprays, and soft gelatin capsules.

12. A method of preventing the spread of a viral infection comprising administering an effective amount of the composition of claim 1 to a body cavity, vessel or a body surface of an individual.

13. The method of claim 12 wherein the formulation is applied to a surface of a body compartment, organ or orifice prior to contact with a virus-containing body fluid.

14. The method of claim 13 wherein the formulation is applied to the vagina, anus, and/or mouth.

15. The method of claim 12, wherein the virus is HIV.

16. The method of claim 12, wherein an effective amount of the formulation is administered to prevent HIV infection in the individual.
